(19) United States
(12) Patent Application Publication
(10) Pub. No.: US 2024/0122624 A1
(43) Pub. Date: Apr. 18, 2024

(54) EXTERNAL FIXATION SYSTEM WITH MULTI-PIN CLAMP AND ATTACHMENT POST

(71) Applicant: GLOBUS MEDICAL, INC., AUDUBON, PA (US)

(72) Inventors: Jesse Rush, Wayne, PA (US); Justin Vent, Drexel Hill, PA (US); Peter Evans, Lafayette Hill, PA (US); David E. Laird, SR., Brandamore, PA (US); Daniel J. Myers, Conshohocken, PA (US); Richard Scheinfield, Philadelphia, PA (US)

(21) Appl. No.: 18/046,704

(22) Filed: Oct. 14, 2022

(51) Int. Cl. A61B 17/64

(52) U.S. Cl. CPC A61B 17/6441; A61B 2017/00477

(57) ABSTRACT

An external fixation system for treatment of bones includes first and second clamps for locking pins that are inserted into bones. At least one clamp includes a socket having a recess. An attachment post includes a distal portion configured to be inserted into the socket recess. A tightening nut is rotationally coupled to a shaft of the attachment post and has an internal threading adapted to be threaded to the external threading of the socket. When the tightening nut is rotated to cause the distal portion of the post to be further inserted into the recess, the sides of the distal portion of the post create an interference fit with the sides of the socket recess to lock the post to the first clamp.

### BACKGROUND

### Field of the Invention

The present invention relates to external fixator systems, and, in particular, to multi-pin clamps and attachment posts threaded into the multi-pin clamps.

### Description of the Related Art

External fixators such as the ARBOR® external fixation system available from Globus Medical, Inc. of Audubon, PA have long been used in trauma incidents as a long-term care solution for reducing fractures and promoting bone healing. Recently, however, external fixators have been used for poly-traumatic patients as a way to stabilize fractures until a more definitive method of fixation can be determined and applied.

In external fixation systems, a multi-pin clamp allows two or more pins to be locked and then connected to other pins in different parts of the body through an angled post threaded into the multi-pin clamp, other clamps and bars, which accommodates various fracture patterns.

In use, the angled post can become loose from the multi-pin clamp due to vibrations or movements of the fixation system. Accordingly, it is desirable to provide a more secure clamping solution for external fixator systems.

### SUMMARY

An external fixation system for treatment of bones includes first and second clamps, and an attachment post configured to be attached to the clamps. The first clamp has a first surface and a socket extending from the first surface, the socket having a recess and an external threading around the recess, the recess having a plurality of sides. The second clamp is configured to be secured to the first clamp by a fastener such as a tightening bolt. The attachment post includes a distal portion having a plurality of sides and is configured to be inserted into the socket recess. The tightening nut is rotationally coupled to the distal portion of the attachment post and has an internal threading which is configured to be threaded to the external threading of the socket. When the tightening nut is rotated to cause the distal portion of the post to be inserted into the recess, the sides of the distal portion of the post create an interference fit with the sides of the socket recess to lock the post to the first clamp.

### BRIEF DESCRIPTION OF THE DRAWINGS

FIG. 1 is a perspective view of a multi-pin clamp in use.

FIG. 2 illustrates a perspective view of a multi-pin clamp 10 according to one aspect of the present invention.

FIG. 3 illustrates a perspective view of an angled post according to one aspect of the present invention.

FIG. 4 is a cross-sectional view of a shaft of the attachment post of FIG. 3.

FIG. 5 is a perspective view of the attachment post of FIG. 3, which has been inserted into the clamp socket of FIG. 2.

FIG. 6 illustrates a cross-sectional view of a clamp socket and attachment post according to another aspect of the present invention.

FIG. 7 is a cross-section view of the clamp socket of FIG. 6.

FIG. 8 is a perspective view of an attachment post according to another aspect of the present invention.

FIG. 9 is a cross sectional view of the attachment post of FIG. 8.

FIG. 10 is a cross-sectional view of a tightening nut according to another aspect of the present invention.

### DETAILED DESCRIPTION

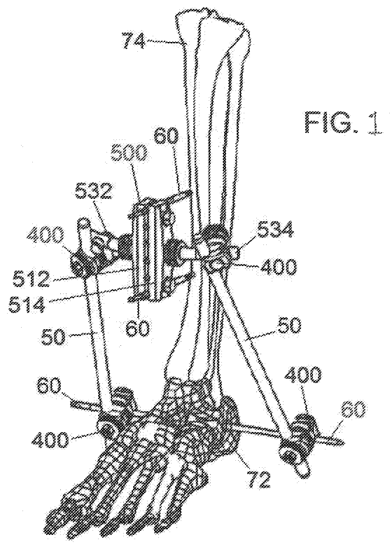

FIG. 1 is a perspective view of a multi-pin clamp 500 in use for locking a plurality of pins 60, such as Schanz pins, that are inserted into a bone 72, 74 of a patient. The multi-pin clamp includes first and second clamps (vice plates) 512, 514 that surround the pins 60. The multi-pin clamp 500 and angled posts 532, 534 (30 degree angled as shown) are configured to be used with one or more external fixator assemblies 400 (four as shown in the present embodiment) to create a construct that is ideal for specific patients. The fixator assemblies 400 and rods 50 are used in conjunction with the multi-pin clamp 500 and angled posts 532, 534 to lock various pins 60 in different parts of the bone. Advantageously, the multi-pin clamp 500 and angled posts 532, 534 help to reduce the number of external fixator assemblies 400 in the overall system, thereby making it less bulky.

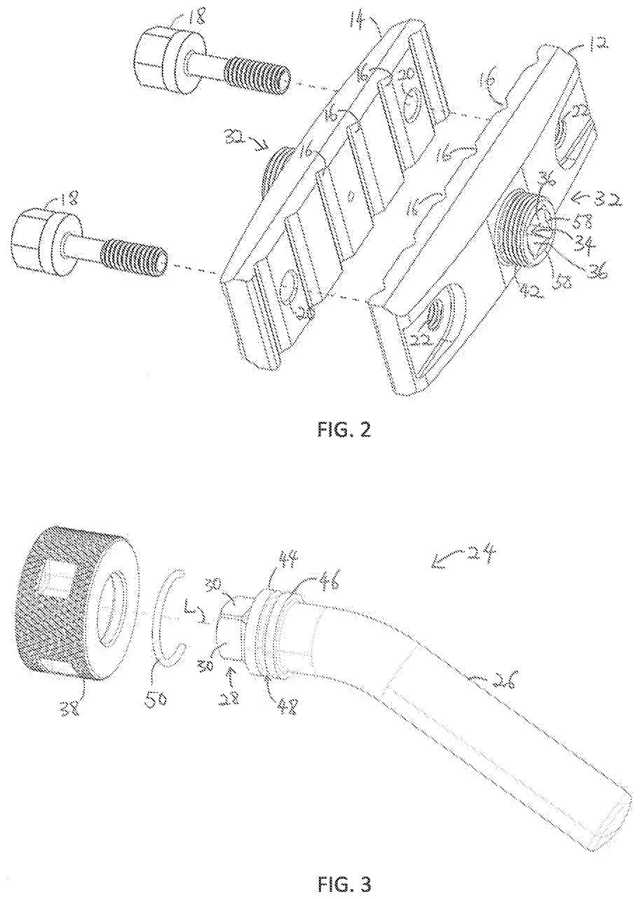

FIG. 2 illustrates a perspective view of a multi-pin clamp 10 according to one aspect of the present invention. The multi-pin clamp 10 includes first and second clamps 12, 14 (e.g., vice plates) capable of receiving one or more pins 60 therethrough and being seated in grooves 16. The respective grooves from the first and second clamps 12, 14 define a channel that is narrowed when the two clamps are brought closer together by fasteners 18 such as tightening bolts. The fasteners 18 are inserted into unthreaded through-openings 20 and are threaded into threaded through-openings 22 to lock the pins 60.

FIG. 3 illustrates a perspective view of an angled post according to one aspect of the present invention. The angled post 24 includes an angled shaft 26 having a distal portion 28 having a plurality of sides 30 (four sides shown in FIG. 3) that are sized to be inserted into a recess 34 of a socket 32 extending from a side surface of the first clamp 12. The recess 34 also has a plurality of sides (four sides shown in FIG. 3) and receives the distal portion 28 of the angled post 24 such that the inserted distal portion cannot rotate in the recess.

A tightening nut 38 is rotationally coupled to the shaft 26 of the post 24 and has an internal threading 40 adapted to be threaded to an external threading 42 of the socket 32. In use, the distal portion 28 is inserted into the socket recess 34. Then, the tightening nut's internal female threading 40 engages the external male threading 42 of the socket 32 and is rotated to further insert the distal portion 28 into the recess 34 in order to lock the attachment post 24 to the first clamp 12.

The shaft 26 of the angled attachment post 24 includes a pair of annular ribs 44, 46 disposed proximally of the distal portion 28 and defining an annular groove 48. A radially compressible retaining ring 50 such as a Circlip is disposed in the annular groove 48 of the angled post 24 and a circumferential recess 52 in the tightening nut 38 to prevent the tightening nut 38 from disengaging from the shaft 26.

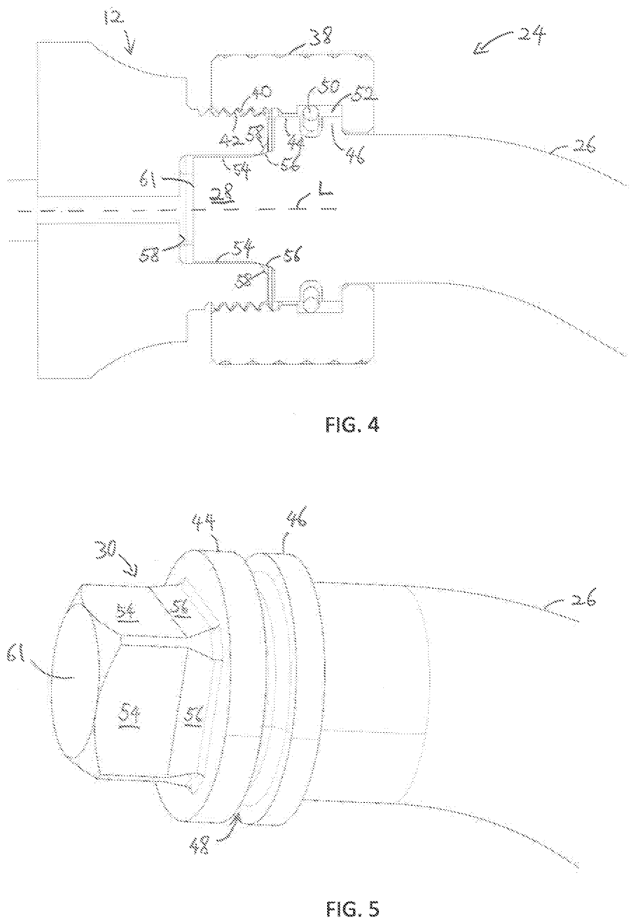

Each side 30 of the distal portion 28 of the tightening nut 38 includes a front portion 54 and a rear portion 56 which has a ramped surface at a selected angle relative to the front portion and a longitudinal axis L defined by the distal portion 28 and the socket 32 (see FIG. 4). When the tightening nut 30 is tightened, the ramped surfaces 56 are pressed against respective chamfered/angled lead-in surfaces 58 of the socket recess 34 for an interference fit (binding effect) to lock the post 24 to the first clamp 12.

The selected angle of the ramped surfaces 56 in the embodiment shown is between 10 degrees and 30 degrees, more preferably 10-20 degrees, which improves the binding effect between the chamfered surfaces 58 and the ramped surfaces 56.

When the tightening nut 38 is rotated to cause the distal portion 28 of the post 24 to be inserted further into the recess 34, the sides 30 of the distal portion of the post creates an interference fit (binding effect) with the sides 36 of the socket recess 34 to lock the post to the first clamp 12.

When the tightening nut 38 is fully tightened, a distal end/surface 61 of the distal portion 28 does not contact a bottom surface 58 of the recess 34. This ensures that the chamfered surfaces 58 and the ramped surfaces 56 create a maximum binding effect.

Although the embodiment as shown in FIG. 3 includes four ramped surfaces 56 on all four sides 30, at least one ramped surface may be acceptable.

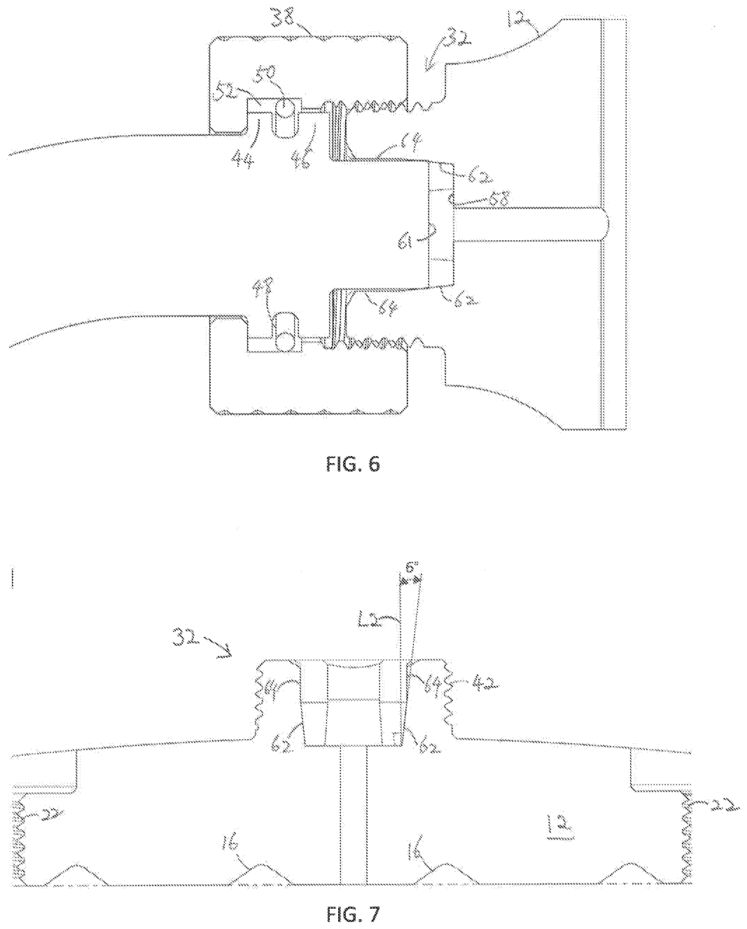

FIG. 6 illustrates a cross-sectional view of a clamp socket 32 according to another aspect of the present invention. Each planar side 36 of the recess 34 includes a vertical planar side 64 along a line L2 perpendicular to the bottom surface 58 and a ramped surface 62 extending at an angle from the vertical side 64 to the bottom surface 58 of the recess 34. The angle of the ramped surface 62 is relative to the longitudinal axis L and to the line L2 and is six degrees in the embodiment shown.

Each ramped surface 62 creates an interference (binding) fit with a respective edge of the distal end 61 of the post distal portion 28. Similar to the embodiment of FIG. 5, when the tightening nut 38 is fully tightened, the distal end 61 of the distal portion 28 is spaced from and does not contact the bottom surface 58 of the recess 34.

Although the embodiment of FIG. 6 includes four angled surfaces 62 in the square-shaped recess 34, one or more of the angled surfaces may be acceptable.

The angle of the ramped surface 62 relative to L2 is important. It should be preferably eight degrees or less, and more preferably between six and eight degrees due to a machine taper effect during manufacture which may cause a cutting tool to bind inside the recess 34.

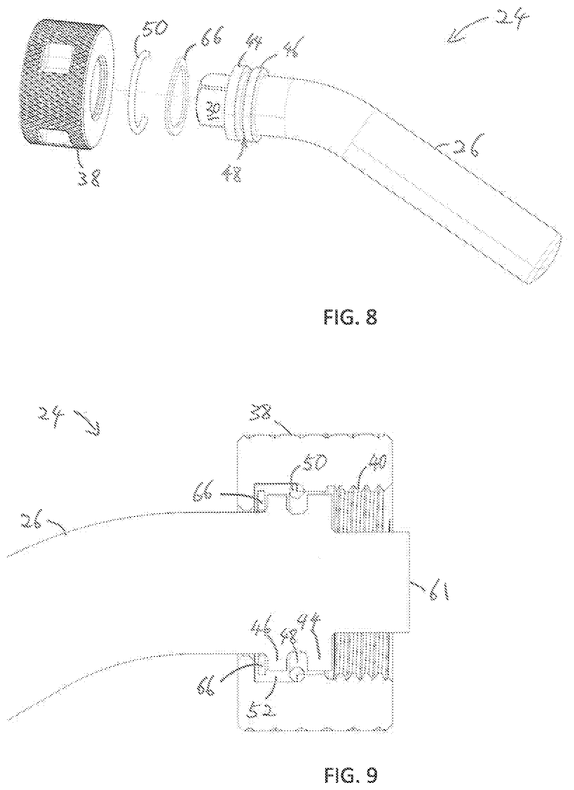

FIG. 8 is a perspective view of another aspect of the present invention and FIG. 9 is a cross sectional view of the attachment post 24 of FIG. 8. In this embodiment, a friction ring 66 is positioned proximally of the annular rib 46 and is seated inside the circumferential recess 52. The friction ring 66 is configured to frictionally bind the attachment post 24 to the socket 32. Any suitable friction ring that provides a frictional force to bind the components may be used including a spring, gap type wave spring, an overlap type wave spring, and PEEK, stainless steel or rubber washer, and the like.

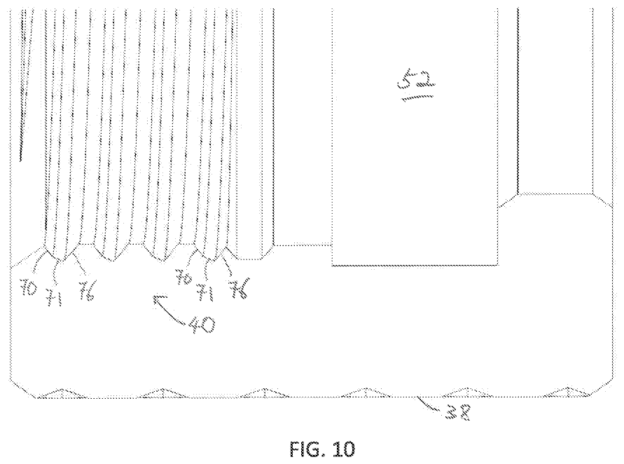

FIG. 10 is a cross-sectional view of a tightening nut according to another aspect of the present invention. Each thread of the female threading 40 of the tightening nut 38 as shown in FIG. 10 includes a declining ramp 70, a wedge ramp 71 following the declining ramp followed by an inclining ramp 76. The wedge ramp 71 has a less steep angle than the declining ramp 70 such that the wedge ramp binds against a standard male external threading 42 of the socket 32 when the tightening nut 38 is tightened. Such female threaded parts can be obtained, for example, from Spiralock Corporation, a Stanley Black & Decker Company, Inc. Company.

Multiple different ways of increasing the frictional force of attachment between the attachment post 24 and the first clamp 12 have been disclosed. Although each embodiment can be used individually, it can be combined with any other embodiments in any combination to produce the desired binding effect depending on application. Thus, the following combination of features is all possible: FIGS. 5 and 6, FIGS. 5 and 8, FIGS. 5 and 10, FIGS. 6 and 8, FIGS. 6 and 10, FIGS. 8 and 10, FIGS. 5, 6 and 8, FIGS. 5, 6 and 10, FIGS. 5, 8 and 10, FIGS. 6, 8 and 10, and FIGS. 5, 6, 8 and 10.

Thus, it will be understood that various changes in the details, materials, and arrangements of the parts which have been described and illustrated in order to explain the nature of this invention may be made by those skilled in the art without departing from the scope of the invention as expressed in the following claims.

What is claimed is:

1. An external fixation system for treatment of bones comprising:
a first clamp having a first surface and a socket extending from the first surface, the socket having a recess and an external threading around the recess, the recess having a plurality of sides;
a second clamp configured to be secured to the first clamp;
an attachment post including:
a distal portion having a plurality of sides and being configured to be inserted into the socket recess; and
a tightening nut rotationally coupled to the distal portion and having an internal threading adapted to be threaded to the external threading of the socket;
wherein when the tightening nut is rotated to cause the distal portion of the post to be inserted into the recess, the sides of the distal portion of the post create an interference fit with the sides of the socket recess to lock the post to the first clamp.

2. The system of claim 1, wherein at least one side of the recess or at least one side of the distal portion of the post has a ramped surface that creates an interference fit between the distal portion of the post and a respective side of the recess.

3. The system of claim 2, wherein at least one side of the recess has the ramped surface that creates an interference fit with a distal end of the distal portion of the post.

4. The system of claim 1, wherein an angle of the ramped surface is eight degrees or less.

5. The system of claim 1, wherein an angle of the ramped surface is between eight degrees and four degrees.

6. The system of claim 1, wherein the recess includes four sides and all of the four sides have a ramped surface that creates an interference fit with a distal end of a respective side of the post.

7. The system of claim 6, wherein an angle of the ramped surface is between eight degrees and four degrees.

8. The system of claim 1, wherein the ramped surface extends from a bottom of the recess.

9. The system of claim 1, wherein at least one side of the distal portion of the post has a ramped surface that creates an interference fit between the ramped surface of the distal portion of the post and a respective side of the recess.

10. The system of claim 9, wherein the post distal portion includes four planar sides and all of the four sides have a ramped surface that creates an interference fit with a respective side of the recess.

11. The system of claim 1, wherein the attachment post includes an annular rib disposed proximally of the distal portion, further comprising a friction ring proximally of the annular rib and configured to frictionally bind the attachment post to the socket.

12. The system of claim 11, wherein the friction ring comprises a wave spring.

13. The system of claim 1, wherein the attachment post includes a second annular rib disposed proximally of the annular rib and proximally of the distal portion to define an annular groove, further comprising a radially flexible retaining ring disposed in the annular groove.

14. The system of claim 1, wherein each thread of the internal threading of the tightening nut includes a declining ramp, a wedge ramp following the declining ramp followed by an inclining ramp, the wedge ramp having a less steep angle than the declining ramp such that the wedge ramp binds against the external threading of the socket when the tightening nut is tightened.

15. An external fixation system for treatment of bones comprising:
a tightening bolt;
a first clamp having a first surface and a socket extending from the first surface and a second surface opposite to the first surface, the socket having a recess and an external threading around the recess, the recess having a plurality of sides defining a perimeter of the recess, the second surface having a plurality of grooves for receiving pins configured to be inserted into a bone;
a second clamp configured to be secured to the first clamp by the tightening bolt to lock the pins to the first and second clamps;
an angled attachment post including:
a distal portion having a plurality of sides and being configured to be inserted into the socket recess; and
a tightening nut rotationally coupled to the attachment post and having an internal threading adapted to be threaded to the external threading of the socket;
wherein when the tightening nut is rotated to cause the distal portion of the post to be inserted into the recess, the sides of the distal portion of the post create an interference fit with the sides of the socket recess to lock the post to the first clamp.

16. The system of claim 15, wherein at least one side of the recess or at least one side of the distal portion of the post has a ramped surface that creates an interference fit between the distal portion of the post and a respective side of the recess.

17. The system of claim 16, wherein at least one side of the recess has the ramped surface that creates an interference fit with a distal end of the distal portion of the post.

18. The system of claim 17, wherein an angle of the ramped surface is between eight degrees and four degrees.

19. The system of claim 15, wherein the angled attachment post includes an annular rib disposed proximally of the distal portion, further comprising a friction ring proximally of the annular rib and configured to frictionally bind the attachment post to the socket.

20. The system of claim 15, wherein each thread of the internal threading of the tightening nut includes a declining ramp, a wedge ramp following the declining ramp followed by an inclining ramp, the wedge ramp having a less steep angle than the declining ramp such that the wedge ramp binds against the external threading of the socket when the tightening nut is tightened.
